(19) United States
(12) Patent Application Publication
(10) Pub. No.: US 2026/0073624 A1
(43) Pub. Date: Mar. 12, 2026

(54) AUTOMATIC CORRECTION OF VIEW FORESHORTENING IN CARDIAC ECHO USING VIEW SYNTHESIS

(71) Applicant: Siemens Medical Solutions USA, Inc., Malvern, PA (US)

(72) Inventors: Tiziano Passerini, Plainsboro, NJ (US); Philippe Bernet, Montévrain (FR)

(21) Appl. No.: 18/882,823

(22) Filed: Sep. 12, 2024

(51) Int. Cl. G06T 15/20; A61B 8/00; A61B 8/08; G06T 5/80; G06T 7/00; G06T 7/11; G06T 7/162; G06T 7/73

(52) U.S. Cl. CPC G06T 15/205; A61B 8/0883; A61B 8/463; A61B 8/5207; G06T 5/80; G06T 7/0012; G06T 7/11; G06T 7/162; G06T 7/75; G06T 2207/10132; G06T 2207/20081; G06T 2207/20084; G06T 2207/30048; G06T 2210/41

(57) ABSTRACT

Systems and methods for automatic correction of view foreshortening in cardiac echo using view synthesis. A trained image segmentation method is used to infer a 2D pose from the input image. The pose is transferred to a new view representing a non-foreshortened view plane. A non-foreshortened image is then rendered.

### FIELD

This disclosure relates to medical imaging.

### BACKGROUND

Cardiac imaging is an important task in assessing ventricular morphology and function. In particular, 2-D echocardiography (2DE) provides for structures to be viewed moving in real time in a cross-section of the heart. 2DE can be used to detect heart diseases or abnormalities, blood clots, tumors, malfunctioning of a heart valve, and abnormality of blood flow within the heart among other conditions. For example, the apical-four-chamber (A4C) view is crucial for many echocardiographic screenings in order to evaluate wall motion and determine left ventricle (LV) function. However, without adequate training and expertise, operators may struggle to orient the probe to ensure all key cardiac measurements are accurate and precise. Even experienced operators may face difficulties acquiring an optimal A4C view due to factors such as obstruction by the patient's ribs, low image contrast, and other non-ideal image conditions. This commonly leads to an acquisition in which the ultrasound probe is not positioned directly on the apex of the heart, and thus the view is foreshortened. In a foreshortened view, the plane is typically oriented at an oblique angle, from the anterior apex to the posterior wall of the atria, leading to a geometric distortion of the left ventricle. The misrepresented left ventricle appears with a deceptively shorter long-axis and a falsely thickened apex.

Foreshortening thus causes incorrect visualization of the heart anatomy and can make it impossible to accurately measure quantities of clinical interest such as ejection fraction and global or segmental strain. Previous attempt to deal with foreshortening have focused on detecting foreshortening in an image after a procedure as a way to exclude foreshortened acquisitions from clinical interpretations. Different approaches have been proposed, based on analysis of the chamber contours, for example by applying shape analysis or direct measurements of geometry properties of the contours. In other implementations, foreshortening was not detected as a distinct feature but rather indirectly, by estimating overall image quality and observing that the presence of foreshortening reduces image quality.

### SUMMARY

By way of introduction, the preferred embodiments described below include methods, systems, instructions, and computer readable media for automatic correction of view foreshortening in cardiac echo using view synthesis.

In a first aspect, a method for automatic correction of view foreshortening in cardiac echo using view synthesis is provided. The method includes acquiring, by a medical imaging device, an image representing a foreshortened view of a heart of a patient; inferring, by a processor, a pose of the heart in the acquired image using a trained image segmentation model and a heart model; generating, by the processor, a non-foreshortened image of the heart of the patient by transferring the inferred pose of the heart to a new synthetic view representing a standard image acquisition plane.

In a second aspect, a system for automatic correction of view foreshortening in cardiac echo using view synthesis is provided. The system includes an ultrasound probe configured to acquire an image of a cardiac region of a patient; an imaging processor configured to generate a non-foreshortened image of the cardiac region of the patient using novel view synthesis, the acquired image, and a heart model; and a display configured to display the non-foreshortened image.

In a third aspect, a non-transitory computer implemented storage medium that stores machine-readable instructions executable by at least one processor for view foreshortening in cardiac echo using view synthesis is provided. The machine-readable instructions include: acquiring a foreshortened image of a cardiac region of a patient using a first view; determining a pose of one or more features of a heart of the patient in the foreshortened image; transferring the pose of the one or more features to a new view of the cardiac region, the new view from a different angle than the first view; and generating a non-foreshortened image from the new view.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the embodiments. Moreover, in the figures, like reference numerals designate corresponding parts throughout the different views.

FIG. 1 depicts an example of a standard imaging plane and a foreshortened imaging plane.

FIG. 2 depicts an example system for automatic correction of view foreshortening in cardiac echo using view synthesis according to an embodiment.

FIG. 3 depicts an example workflow for automatic correction of view foreshortening in cardiac echo using view synthesis according to an embodiment.

FIG. 4 depicts an example artificial neural network.

FIG. 5 depicts an example convolutional neural network.

FIG. 6 depicts an example flowchart for automatic correction of view foreshortening in cardiac echo using view synthesis according to an embodiment.

FIG. 7 depicts an example of foreshortened images and non-foreshortened images.

### DETAILED DESCRIPTION

Embodiments described herein provide systems and methods for view foreshortening in cardiac echo using view synthesis. A foreshortened image is acquired from a first view. The pose of the heart is determined in the foreshortened image using a segmentation method and heart model. The pose is transferred to a new non-foreshortened view. A synthetic non-foreshortened image is rendered and displayed.

A cardiac ultrasound or echocardiogram is an ultrasound procedure that allows an operator to visualize and evaluate how the heart of a subject is functioning. While there are different types of cardiac ultrasound procedures, the cardiac ultrasound technique referred to as “Transthoracic” or Transesophageal” echocardiography is used as the primary example in this document. Transthoracic Echocardiography (TTE) is a non-invasive cardiac ultrasound acquisition performed on the patient's chest. There are a total of 4 chambers in the heart: Right Atrium (RA), Right Ventricle (RV), Left Atrium (LA), and Left Ventricle (LV). These are separated by the atrioventricular valves: Tricuspid Valve between RA and RV, Mitral Valve between LA and LV. With TTE, by placing the ultrasound transducer/probe at different locations and at different angles, the operator is able to visualize all of these structures. Different cardiac ultrasound views of the heart may include, for example, the Parasternal Long Axis, Parasternal Short Axis, Apical 4 Chamber, Subxiphoid (Subcostal), and IVC Views. The Apical 4 Chamber (A4C), in particular, is one of the most important views for hemodynamic assessment of the heart. The A4C view allows an operator to visualize diastolic dysfunction, valvular regurgitation, cardiac output, etc. Unfortunately, the A4C view may be one of the most challenging views to obtain due to the precise orientation required for positioning the transducer/probe.

The A4C view may be generated by placing the transducer/probe so that the view intersects the LV apex, the mitral, and the tricuspid valve centers. However, if the probe is held at the wrong angle, for example offset by 5, 10, or 20 degrees from the optimal plane, the view may be foreshortened, e.g., other portions of the ventricular wall will appear in the image. Foreshortening of apical views is a common problem in echocardiography. It results in an abnormally thick false apex and a shortened left ventricular (LV) long axis. Foreshortened echocardiographic views are characterized by an imaging plane that, for example, transects the heart above and anterior of the true apex leading to a geometric distortion of the image of the left ventricle. As a result, the long axis of the left ventricle appears shorter and the false apex is thicker and apparently hyper-contractile resulting in an overestimation of both global and regional left ventricular (LV) function and an underestimation of LV volume and length. Furthermore, assessment of apical geometry and function are hindered.

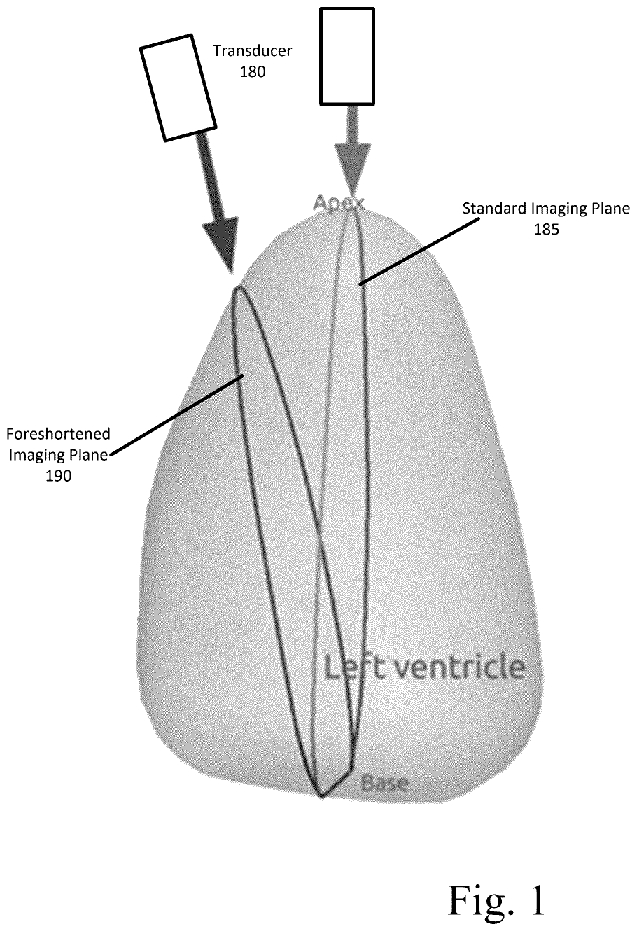

FIG. 1 depicts an example of imaging planes that can lead to foreshortening. In FIG. 1, a transducer 180 is placed at an angle to the left ventricle. When the angle is correct, the resulting view, the standard imaging plane 185 cuts through the true apex of the left ventricle. When the angle is incorrect, e.g., offset, the foreshortened imaging plane 190 does not. Due to the incorrect orientation, using the foreshortened imaging plane 190 for volume estimation will result in an underestimation of the left ventricular volume, for example as depicted in FIG. 1, the area in the slice is, for example, smaller than the area in the slice provided by the standard imaging plane 185.

Current methods are focused on detecting foreshortening. In particular, several deep learning methods have been used to detect whether an image is foreshortened or not. In an example, a classification network may be used to classify cardiac views such as A4C, A2C, apical long axis, parasternal short axis, parasternal long axis, subcostal four chamber, subcostal vena cava, and unknown views. A system then determines measurements such as volume and EF for example using end-diastole (ED) and end-systole (ES) frames. The measurements are then analyzed to determine if a particular view is foreshortened or not. In another example, a network may be trained using synthetic apical-four-chamber (A4C) views with matching ground truth foreshortening labels. A statistical shape model of the four chambers of the heart may be used to synthesize idealized A4C views with varying degrees of foreshortening. Contours of the left ventricular endocardium may be segmented in the images, and a partial least squares (PLS) model may then be trained to learn the morphological traits of foreshortening. While identification of foreshortened views is an important task, it does not solve the underlying problems, that for example include how to prevent apical foreshortening by guiding the image acquisition and how to compensate for existing apical foreshortening in previously acquired images to still produce clinically meaningful measurements.

Embodiments described herein provide systems and methods for automatic correction of view foreshortening in cardiac echo using view synthesis. In an embodiment, view synthesis is used to generate a synthetic non-foreshortened view from a real foreshortened view. The view synthesis may be performed in real time, so that synthesized views may be displayed to an operator during the acquisition of the images. By assessing the discrepancy between the actual view and the synthesized, non-foreshortened view, the operator may be able to modify the probe position and orientation until a non-foreshortened view is obtained. In addition, the synthesized non-foreshortened view may be utilized instead or in combination with the actual view for performing measurement of clinically relevant quantities such as chamber volume, ejection fraction, global strain, and longitudinal strain.

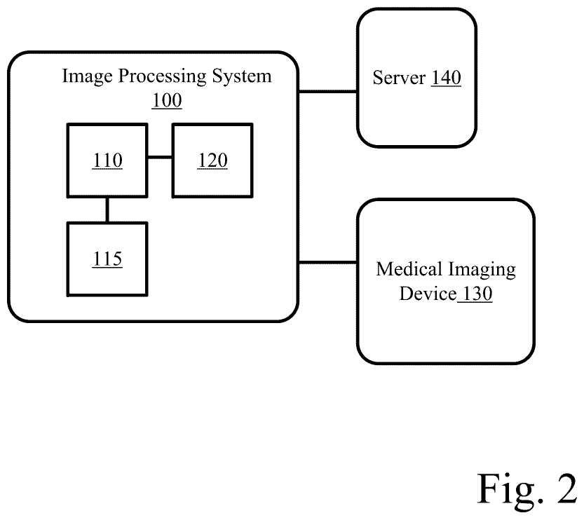

FIG. 2 depicts a system for automatic correction of view foreshortening in cardiac echo using view synthesis. The system includes an image processing system 100, a medical imaging device 130, and optionally a server 140. The medical imaging device 130 is configured to acquire two dimensional image(s). The image processing system 100 includes a processor 110, a memory 120, and a display 115. The image processing system 100 may be included with or coupled to the medical imaging device 130. The image processing system 100 is configured to automatically correct an acquired foreshortening view using view synthesis. The image processing system 100 may also be configured to train or store one or more machine learned models for these tasks. The server 140 may be configured to perform any of the tasks of the image processing system 100 including processing and/or storing of the data and models. The server 140 may be or may include a cloud-based platform. Additional, different, or fewer components may be provided. For example, a computer network is included for remote processing of locally captured ultrasound data, for example by the server 140. As another example, a user input device (e.g., keyboard, buttons, sliders, dials, trackball, mouse, or other device) is provided for user alteration or placement of one or more markers.

For the medical imaging device 130, the example used herein is in an ultrasound context, but other types of scanners may be used (e.g., MR, PET, SPECT, or other medical imaging devices) that generate two dimensional foreshortened images. In an embodiment, the ultrasound system 130 is configured to generate two dimensional ultrasound images of a patient during an imaging procedure. The ultrasound imaging process (also called sonography) uses high-frequency sound waves to produce real-time images of organs, tissues, blood flow, and other patient features. Echocardiography, also known as cardiac ultrasound, is the use of ultrasound to examine the heart. Echocardiography is routinely used in the diagnosis, management, and follow-up of patients with any suspected or known heart diseases. Different ultrasound techniques may be used to perform echocardiography including transthoracic echocardiogram (TTE), transesophageal echocardiogram (TEE), and Intracardiac Echocardiogram (ICE) among others. TTE is a non-invasive procedure where a transducer (or probe) is placed on the chest of the patient. Image(s) are then acquired. The embodiments described herein use TTE as an example, but other ultrasound imaging techniques and other patient regions may be used. In an embodiment, the medical imaging device 130 is configured to acquire two-dimensional images (slices) of a region of interest of a patient, for example, of a cardiac region of the patient. Due to improper use such as an incorrect placement or orientation of the transducer/probe by the operator, the two-dimensional images may be foreshortened and thus lead to erroneous metrics and/or an inaccurate diagnosis.

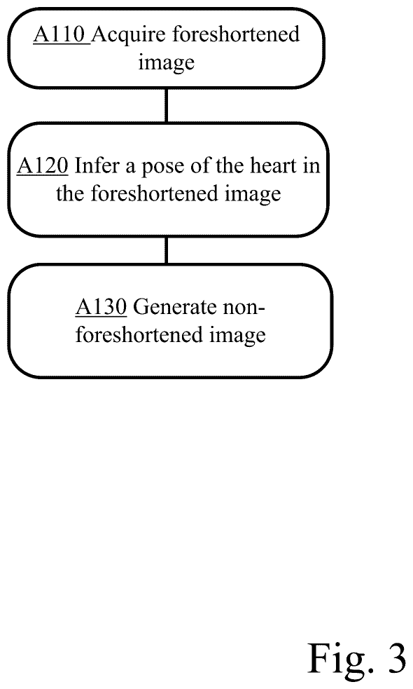

In an embodiment, the processor 110 is configured to use novel view synthesis to generate a synthetic non-foreshortened view when provided with a foreshortened view. The processor 110 is a general processor, digital signal processor, graphics processing unit, application specific integrated circuit, field programmable gate array, artificial intelligence processor, digital circuit, analog circuit, combinations thereof, or other now known or later developed device for automatic correction of view foreshortening in cardiac echo using view synthesis, among other processes described below. The processor 110 is a single device, a plurality of devices, or a network. For more than one device, parallel or sequential division of processing may be used. Different devices making up the processor 110 may perform different functions. In one embodiment, the processor 110 is a control processor or other processor of the medical imaging device 130. In other embodiments, the processor 110 is part of a separate workstation or computer. The processor 110 operates pursuant to stored instructions to perform various acts described herein. The processor 110 is configured by software, design, firmware, and/or hardware to perform any or all of the acts of FIGS. 3-6 and any other computations described herein.

Image data, the machine trained networks, training data, computed metrics, and other data may be stored in the memory 120. The memory 120 may be or include an external storage device, RAM, ROM, database, and/or a local memory (e.g., solid state drive or hard drive). The same or different non-transitory computer readable media may be used for the instructions and other data. The memory 120 may be implemented using a database management system (DBMS) and residing on a memory 120, such as a hard disk, RAM, or removable media. Alternatively, the memory 120 is internal to the processor 110 (e.g., cache). The instructions for implementing the processes, methods, and/or techniques discussed herein are provided on non-transitory computer-readable storage media or memories, such as a cache, buffer, RAM, removable media, hard drive, or other computer readable storage media (e.g., the memory 120). The instructions are executable by the processor 110 or another processor. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the instructions set, storage media, processor 110 or processing strategy and may be performed by software, hardware, integrated circuits, firmware, micro code, and the like, operating alone or in combination. In one embodiment, the instructions are stored on a removable media device for reading by local or remote systems. In other embodiments, the instructions are stored in a remote location for transfer through a computer network. In yet other embodiments, the instructions are stored within a given computer, CPU, GPU, or system. Because some of the constituent system components and method steps depicted in the accompanying figures may be implemented in software, the actual connections between the system components (or the process steps) may differ depending upon the manner in which the present embodiments are programmed.

Novel view synthesis (also called “view synthesis”) is the task of generating an image of a scene from a previously unseen perspective. Novel view synthesis is a fundamentally ill-posed problem in that the solution is highly sensitive to changes in the data. To provide novel view synthesis, the problem is separated into two parts. The first part includes solving an inverse graphics problem, requiring a deep understanding of the scene, while the second relies on image synthesis to generate realistic images using this understanding of the scene. In an example, an encoder-decoder architecture may be used, where the encoder solves an image understanding problem via some latent representation, which is subsequently used by the decoder for image synthesis.

As used herein, the term “scene” refers to the pose of an object in a 3D world. A view is the image of a scene obtained by projection onto a 2D plane. Novel view synthesis generates a new synthetic view, starting from one or more given images of the scene. In the context of cardiac echocardiography as described herein, the scene is the beating heart—in certain embodiments, just the left ventricle. One or more views may be acquired, defined on planes that slice the heart according to predefined rules (e.g., for apical views the plane includes the apex of the chamber in addition to specific structures at the base of the chamber, for example corresponding to valvular structures). The image of the scene is not generated by projection, but rather by sonography, with high-frequency sound waves traveling through the scene along multiple beams forming the plane of the view. Given an image of the scene, for instance the left ventricle observed in an apical 4-chamber plane, the embodiments synthesize novel views including, for example, a non-foreshortened version of the current view and/or a non-foreshortened version of additional apical views, for example, apical 2- and 3-chamber views. View synthesis may be performed in real time, so that the synthesized views may be displayed to the operator during the acquisition of the images. By assessing the discrepancy between the actual view and the synthesized, non-foreshortened view, the operator may be able to modify the probe position and orientation until a non-foreshortened view is obtained. View synthesis may be performed offline as well, for example, using previously acquired images. In real-time or offline, the synthesized non-foreshortened view may be utilized instead or in combination with the actual view for performing measurements of clinically relevant quantities such as chamber volume, ejection fraction, global and longitudinal strain.

In an embodiment, view synthesis is performed by the processor 110 by jointly solving an inverse graphics problem (understanding a scene from a view) and image synthesis problem. The processor 110 is configured to model the heart as a graph, with N vertices and M edges. The vertices represent location of junctions between different sections of the heart. The edges can include leaflets of the heart valves (tricuspid, aortic, mitral), segments of each heart chamber (e.g., 18 segments in the left ventricle as commonly adopted in clinical practice), and/or proximal portions of arteries and veins attached to the heart (aorta, pulmonary artery, vena cava, pulmonary veins). The heart model is used to determine the pose of the image, for example by matching landmarks or boundaries in the image with associated edges in the heart model. A side product of this approach is the estimation of the 3D pose of the heart model, for each individual temporal frame in the sequence of acquired images, therefore allowing the processor 110 to estimate the motion of the heart model. This may be used to assist in estimating quantities of clinical interest, such as chamber volumes, ejection fraction, global and segmental strain.

To generate a non-foreshortened view, the processor infers the pose and appearance information of the heart from the acquired image. The pose is then transferred to a novel view along with the appearance, from which the processor renders diffuse primitives, using a realistic model. The primates represent diffuse three dimensional shapes, for example, semantically meaningful structural locations in the heart. The processor then may use an encoder-decoder architecture to generate a novel realistic non-foreshortened image. In an embodiment, the framework may be trained end-to-end, optimizing both image reconstruction quality and pose estimation.

In an embodiment, the processor is configured to use a trained image segmentation method to infer the 2D pose from the input image. The image segmentation method is trained to recognize in a given view the visible anatomical structures as described by the heart model for each edge in the graph. For example, the acquired image may be segmented and the leaflets and the segments in the left ventricle are identified. The identified leaflets and the segments may be matched with the respective leaflets and segments from the heart model to determine the orientation of the slice, e.g., the pose of the heart in the view.

The processor 110 is configured to generate a new image of the heart as seen from another view by transferring the determined pose to an image plane that produces a non-foreshortened view. Differential rendering may be used to render the heart model into the newly generated view and a trained model may apply the appearance to the rendered image. Differentiable rendering (DR) is a technique that renders a scene, then obtains derivatives of the output pixel values with respect to continuous inputs: rendering primitives, camera intrinsics, lighting, texture values, etc. DR computes derivative images from an original image. The processor is configured to render a simplified skeletal structure of the diffuse primitives obtained from the pose. Each primitive may be understood as an anisotropic Gaussian defined by its location and shape, and the rendering operation is the process of integrating along each ray. Occlusions are handled by a smooth aggregation step. The renderer is differentiable with respect to each input parameter, as the rendering function is itself a composition of differentiable functions.

In an embodiment, selection of the image plane that produces a non-foreshortened view may be based on pre-defined rules. For instance, an apical 4-chamber view with no apical foreshortening of the left ventricle uses the plane defined by three points: left ventricular apex, middle of the mitral anterior leaflet, middle of the mitral posterior leaflet. The selected plane may be defined for a specific temporal frame (e.g., end-diastolic temporal frame) and kept constant for the entire sequence or may be updated for each temporal frame.

In an embodiment, view synthesis is based on the use of a sonography simulator. In this case, a heart model is obtained by segmentation/tracking of cardiac structures in time-resolved 4D cardiac images followed by shape analysis. The motion of the heart in any apical plane may be modeled with manifold learning techniques applied to the heart contour on the plane. A discrete number of planes may be pre-defined and separate motion models can be defined for each plane. For a given input time sequence of 2D echocardiography images, automatic segmentation/tracking is applied to generate a time sequence of contours. Based on similarity between the actual contours and the motion models, one motion pattern is selected as the most similar to the one observed in the images. The corresponding plane is selected as part of the computation of the similarity between the motion model and the actual contour sequence. The 3D heart model is scaled and aligned (registered) with the acquired image(s) based on the selected plane. The 3D model may be used to derive estimated of measurement of clinical utilities such as chamber volumes, ejection fraction, global and segmental strain. A sonography simulator may be used to produce a non-foreshortened view using the aligned heart model as input to select the optimal plane.

Using novel view synthesis, the processor 110 produces the non-foreshortened view for the selected plane. The non-foreshortened view is rendered to match the appearance of the other views. The rendered view is then analyzed and/or provided to the operator. The display 115 may be configured to display or otherwise provide the images to the user. The display 115 is a CRT, LCD, projector, plasma, printer, tablet, smart phone or other now known or later developed display device for displaying the output. In an embodiment, the non-foreshortened view is displayed to the operator during the acquisition allowing the operator to use the non-foreshortened view as a reference to visually determine whether or not the amount of foreshortening in the actual acquisition is acceptable. For example, an outline of the components of the non-foreshortened view and the real time acquired (foreshortened) view may be overlaid on one another so that the operator can move the transducer until the real image provides the correct optimal view. In another embodiment, the non-foreshortened view (and potentially the additional non-foreshortened views) may be input into a system trained to perform measurements (such as auto-contouring, volume computation, speckle tracking and strain computation) and the measurements are stored and/or returned to the operator. The actually acquired view that includes foreshortening may also be input to the measurement system. If the measurements obtained on the non-foreshortened view are available, a measurement error is defined as the difference between the value estimated on the foreshortened view and the value estimated on the non-foreshortened view, normalized by the value estimated on the non-foreshortened view. The error may be provided to the user as a measure of sub-optimality of the acquired view.

In an embodiment, when the method is applied on previously acquired images, the measurement error may also be used as an exclusion criterion for images with excessive foreshortening. In that case, if the measurement error exceeds a pre-defined threshold, the image is excluded from further processing. The method provides a synthesized non-foreshortened view based on the input image, which may be used instead or in combination with the actual image. In one embodiment, the clinical measurements are defined as a linear combination between the measurement computed on the foreshortened image and the one computed on the non-foreshortened view. The weights of the linear combination can be pre-set to default values or can be adjusted based on the expected uncertainty in the final output. Each weight is between 0 and 1 and the sum of the weights is 1. A higher weight on the measurement obtained from the actual view discounts the accuracy of the synthesized non-foreshortened view, and conversely for a lower weight.

In addition to displaying the images and/or information, the processor 110 may further be configured to define the modifications required in the acquisition plane to modify the actual view to a non-foreshortened one. In an embodiment, the operator is notified that the actual view is foreshortened both based on a visual impression (visual comparison between the actual view and the synthetized non-foreshortened one) and based on the measurement error. The operator may then modify the position and orientation of the ultrasound probe until the visual impression shows no foreshortening and/or the measurement error is below a pre-defined threshold.

FIG. 3 depicts a method for automatic correction of view foreshortening in cardiac echo using view synthesis. The acts are performed by the system of FIGS. 2 and 4-6, other systems, a workstation, a computer, and/or a server. Additional, different, or fewer acts may be provided. The acts are performed in the order shown (e.g., top to bottom) or other orders. Certain acts may be omitted or changed depending on the results of the previous acts and the status of the patient. In an embodiment, from an input image, 3D heart features are inferred and appearance vectors are estimated. The pose is transferred from the input view to the novel view. The appearance vectors are rendered onto a high-dimensional latent image. From the latent image, the output image is synthesized.

At act A110, an image representing a foreshortened view of a heart of a patient is acquired. In an embodiment, the method is performed by a medical diagnostic ultrasound scanner 130. Alternative imaging modalities may be used that acquire two dimensional images. The transducer scans a plane. The scan plane is oriented based on a position of the catheter. As the transducer is moved (e.g., translates or rotates), different scan planes may be scanned. Each scan generates a frame of data representing the scan plane at that time. The frame of ultrasound data may be scalar values or display values (e.g., RGB) in a polar coordinate or Cartesian coordinate format. The frame of ultrasound data may be a B-mode, color flow, or other ultrasound image. The image may be of a foreshortened view. In a foreshortened view, the apical region appears rounded. When echocardiography is performed correctly, the apical region appears with a bullet-shape. Foreshortening of apical views results in an abnormally thick false apex and a shortened left ventricular (LV) long axis. The image may include a plurality of pixels, each of which represents tissues, blood, or other features of the cardiac region of the patient.

At act A120, a pose of the heart in the acquired image is inferred using a trained image segmentation model and the heart model. The heart model may be generated prior to or subsequent to the acquisition of the image at act A110. In an embodiment, the heart is modelled as a graph, with N vertices and M edges. The vertices represent location of junctions between different sections of the heart. The edges can include leaflets of the heart valves (tricuspid, aortic, mitral), segments of each heart chamber (e.g., 18 segments in the left ventricle as commonly adopted in clinical practice), proximal portions of arteries and veins attached to the heart (aorta, pulmonary artery, vena cava, pulmonary veins).

In an embodiment, the known 3D structure of the heart from the heart model is used, for example where identified points, features, and landmarks etc. from the acquired image are used to determine how the slice is orientated. The identified points, features, and landmarks in the acquired image may be identified by segmenting the image. In an embodiment, an image segmentation method is trained to recognize in a given view all the visible anatomical structures that are included in the heart model. For example, the leaflets of the heart valves (tricuspid, aortic, mitral), segments of each heart chamber (e.g., 18 segments in the left ventricle as commonly adopted in clinical practice), proximal portions of arteries and veins attached to the heart (aorta, pulmonary artery, vena cava, pulmonary veins) may be identified in the acquired image. The identified anatomical structures may then be aligned/matched with the heart model to determine the orientation/angle of the acquired slice. When the slice is acquired at more than a predetermined angle (for example 5, 10, or 20 degrees) from the standard/optimal view, the slice may provide a significantly foreshortened view that may affect measurements of the heart function. The larger the angle between the acquired slice and the standard view, the more the structures of the heart are misshaped. By segmenting the acquired slice and attempting to fit it into the heart model, the system can determine the orientation/angle of the slice. Different methods may be used for the segmentation and/or classification of the acquired image slice. For example, segmentation may be thresholding-based, region-based, shape-based, model based, neighboring based, and/or machine learning-based among other segmentation techniques. Thresholding-based methods segment the image data by creating binary partitions based on image attenuation values, as determined by the relative attenuation of structures on the images. Region-based segmentation compares one pixel in an image to neighboring pixels, and if a predefined region criterion (e.g., homogeneity) is met, then the pixel is assigned to the same class as one or more of its neighbors. Shape-based techniques use either an atlas-based approach or a model-based approach to find a boundary of the organ. Model-based methods use prior shape information, similar to atlas-based approaches; however, to better accommodate the shape variabilities, the model-based approaches may fit either statistical shape or appearance models of the organ to the image by using an optimization procedure. Neighboring anatomy-guided methods use the spatial context of neighboring anatomic objects. In machine learning-based methods, boundaries are predicted on the basis of the features extracted from the image data. A SoftMax layer or other classification technique, for example, may be provided to classify/identify the segmented pixels/boundaries/tissues etc., in particular, the leaflets of the heart valves (tricuspid, aortic, mitral), segments of each heart chamber (e.g., 18 segments in the left ventricle as commonly adopted in clinical practice), and proximal portions of arteries and veins attached to the heart (aorta, pulmonary artery, vena cava, pulmonary veins).

Machine learning for image segmentation may be done by extracting a selection of features from input images. These features may include, for example, pixel gray levels, pixel locations, image moments, information about a pixel's neighborhood, etc. A vector of image features is then fed into a learned classifier which classifies each pixel of the image into a class. The parameters of the classifier are learned automatically by giving the classifier input images for which the ground truth classification results is known, for example annotated images. The output of the model is then compared to the ground truth, and the parameters of the model are adjusted so that the model's output better matches the ground truth value. This procedure is repeated for a large amount of input images, so that the learned parameters generalize to new, unseen examples. Deep learning may also be used for segmentation (and other tasks described herein), for example using a neural network. Deep learning-based image segmentation may be done, for example, using a convolutional neural network (CNN). The convolutional neural network includes a layered structure where series of convolutions are performed on an input image. Kernels of the convolutions are learned during training. The convolution results are then combined using a learned statistical model that outputs a segmented image. The image segmentation method may be trained using synthetically generated views, and labels generated by intersecting the heart model with the plane representing the image acquisition plane.

In an embodiment, given a 2D image of a heart, 3D pose estimation produces a 3D view that matches the spatial position of the depicted view. The input is a series of 2D points x∈R^{2×n}, and the output is a series of points in 3d space y∈R^{3×n}. The model is trained to learn a function f*:R^{2×n}→R^{3×n} that minimizes the prediction error over a dataset of N poses. The pose is first localized in the 2D space, then regressed in 3D. Simultaneously appearance vectors may also be inferred from the input image. Primitives, naturally deducted from the pose, are rendered in the novel viewpoint using intrinsic parameters and distortion coefficients of the ultrasound imaging procedure. The output image may then be generated as described below using a high-dimensional feature image alongside with the appearance vectors.

Embodiments leverage the power of artificial intelligence (AI) to provide a more accurate and efficient imaging procedure. In an embodiment, the system 100 is configured to train and/or implement one or more machine learned networks, for example that make up the segmentation network, differentiable renderer, etc. The machine learned network(s) or model(s) may include a neural network that is defined as a plurality of sequential feature units or layers. Sequential is used to indicate the general flow of output feature values from one layer to input to a next layer. The information from the previous layer is fed to the next layer, and so on until the final output. The layers may only feed forward or may be bi-directional, including some feedback to a previous layer. The nodes of each layer or unit may connect with all or only a sub-set of nodes of a previous and/or subsequent layer or unit. Skip connections may be used, such as a layer outputting to the sequentially next layer as well as other layers. Rather than pre-programming the features and trying to relate the features to attributes, the deep architecture is defined to learn the features at different levels of abstraction based on the input data. The features are learned to reconstruct lower-level attributes (i.e., attributes at a more abstract or compressed level). Each node of the unit represents a feature. Different units are provided for learning different features. Various units or layers may be used, such as convolutional, pooling (e.g., max pooling), deconvolutional, fully connected, or other types of layers. Within a unit or layer, any number of nodes is provided. For example, 100 nodes are provided. Later or subsequent units may have more, fewer, or the same number of nodes. Different configurations of networks may be used for different applications. Different training mechanisms and training data may be used for different applications.

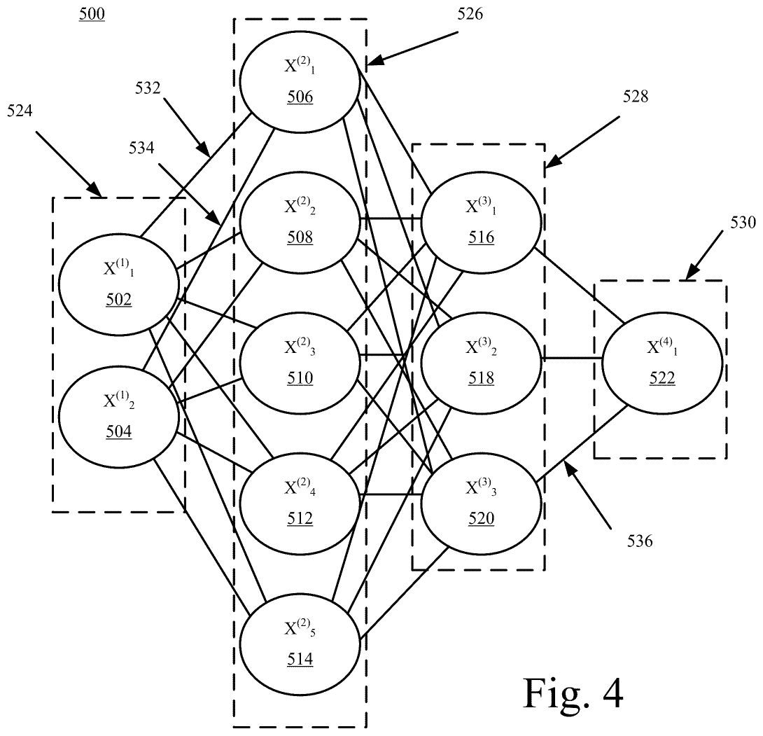

FIG. 4 shows an embodiment of an artificial neural network 500, in accordance with one or more embodiments. Alternative terms for “artificial neural network” are “neural network”, “artificial neural net” or “neural net”. The artificial neural network 500 may be used in part in, for example, the one or more machine learning based networks utilized for segmentation, pose detection, rendering, etc.

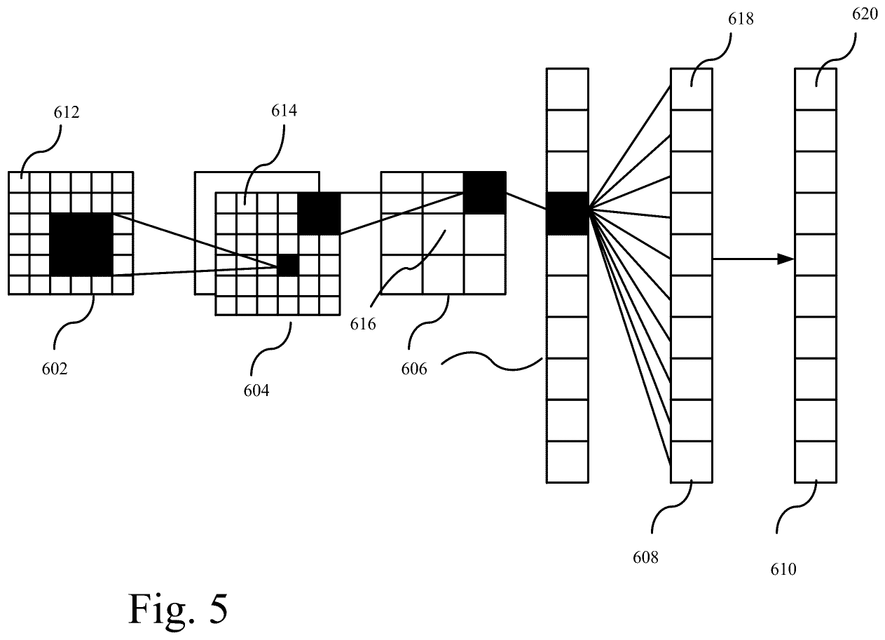

The artificial neural network 500 includes nodes 502-522 and edges 532, 534, . . . , 536, wherein each edge 532, 534, . . . , 536 is a directed connection from a first node 502-522 to a second node 502-522. In general, the first node 502-522 and the second node 502-522 are different nodes 502-522, it is also possible that the first node 502-522 and the second node 502-522 are identical. In FIG. 5, the edge 532 is a directed connection from the node 502 to the node 506, and the edge 534 is a directed connection from the node 504 to the node 506. An edge 532, 534, . . . , 536 from a first node 502-522 to a second node 502-522 is also denoted as “ingoing edge” for the second node 502-522 and as “outgoing edge” for the first node 502-522.

In this embodiment, the nodes 502-522 of the artificial neural network 500 may be arranged in layers 524-530, wherein the layers may include an intrinsic order introduced by the edges 532, 534, . . . , 536 between the nodes 502-522. In particular, edges 532, 534, . . . , 536 may exist only between neighboring layers of nodes. In the embodiment shown in FIG. 5, there is an input layer 524 including only nodes 502 and 504 without an incoming edge, an output layer 530 including only node 522 without outgoing edges, and hidden layers 526, 528 in-between the input layer 524 and the output layer 530. In general, the number of hidden layers 526, 528 may be chosen arbitrarily. The number of nodes 502 and 504 within the input layer 524 usually relates to the number of input values of the neural network 500, and the number of nodes 522 within the output layer 530 usually relates to the number of output values of the neural network 500.

In particular, a (real) number may be assigned as a value to every node 502-522 of the neural network 500. Here, x⁽ⁿ⁾ᵢ denotes the value of the i-th node 502-522 of the n-th layer 524-530. The values of the nodes 502-522 of the input layer 524 are equivalent to the input values of the neural network 500, the value of the node 522 of the output layer 530 is equivalent to the output value of the neural network 500. Furthermore, each edge 532, 534, . . . , 536 may include a weight being a real number, in particular, the weight is a real number within the interval [−1, 1] or within the interval [0, 1]. Here, w^{(m,n)}_{i,j} denotes the weight of the edge between the i-th node 502-522 of the m-th layer 524-530 and the j-th node 502-522 of the n-th layer 524-530. Furthermore, the abbreviation w⁽ⁿ⁾_{i,j} is defined for the weight w^{(n,n+1)}_{i,j}.

In particular, to calculate the output values of the neural network 500, the input values are propagated through the neural network. In particular, the values of the nodes 502-522 of the (n+1)-th layer 524-530 may be calculated based on the values of the nodes 502-522 of the n-th layer 524-530 by

$${x}_{j}^{( n + 1 )} = f ( {\sum }_{i} {x}_{i}^{( n )} \cdot {w}_{i , j}^{( n )} ) .$$

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 524 are given by the input of the neural network 500, wherein values of the first hidden layer 526 may be calculated based on the values of the input layer 524 of the neural network, wherein values of the second hidden layer 528 may be calculated based in the values of the first hidden layer 526, etc.

In order to set the values w^{(m,n)}_{i,j} for the edges, the neural network 500 has to be trained using training data. In particular, training data includes training input data and training output data (denoted as tᵢ). For a training step, the neural network 500 is applied to the training input data to generate calculated output data. In particular, the training data and the calculated output data include a number of values, said number being equal with the number of nodes of the output layer.

In particular, a comparison between the calculated output data and the training data is used to recursively adapt the weights within the neural network 500 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers δ⁽ⁿ⁾ⱼ may be recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on δ⁽ⁿ⁺¹⁾ⱼ, if the (n+1)-th layer is not the output layer, and

$${δ}_{j}^{( n )} = ( {x}_{k}^{( n + 1 )} - {ty}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 530, wherein f′ is the first derivative of the activation function, and y⁽ⁿ⁺¹⁾ⱼ is the comparison training value for the j-th node of the output layer 530.

FIG. 5 shows a convolutional neural network 600, in accordance with one or more embodiments. Machine learning networks described herein, such as, e.g., for the segmentation, pose detection, rendering, etc. may be implemented using the convolutional neural network 600.

In the embodiment shown in FIG. 5, the convolutional neural network 600 includes an input layer 602, a convolutional layer 604, a pooling layer 606, a fully connected layer 608, and an output layer 610. Alternatively, the convolutional neural network 600 may include several convolutional layers 604, several pooling layers 606, and several fully connected layers 608, as well as other types of layers. The order of the layers may be chosen arbitrarily, usually fully connected layers 608 are used as the last layers before the output layer 610.

In particular, within a convolutional neural network 600, the nodes 612-620 of one layer 602-610 may be considered to be arranged as a d-dimensional matrix or as a d-dimensional image. In particular, in the two-dimensional case the value of the node 612-620 indexed with i and j in the n-th layer 602-610 may be denoted as x⁽ⁿ⁾_{[i,j]}. However, the arrangement of the nodes 612-620 of one layer 602-610 does not have an effect on the calculations executed within the convolutional neural network 600 as such, since these are given solely by the structure and the weights of the edges.

In particular, a convolutional layer 604 is characterized by the structure and the weights of the incoming edges forming a convolution operation based on a certain number of kernels. In particular, the structure and the weights of the incoming edges are chosen such that the values x⁽ⁿ⁾ₖ of the nodes 614 of the convolutional layer 604 are calculated as a convolution x⁽ⁿ⁾ₖ=Kₖ*x⁽ⁿ⁻¹⁾ based on the values x⁽ⁿ⁻¹⁾ of the nodes 612 of the preceding layer 602, where the convolution * is defined in the two-dimensional case as:

$${x}_{k}^{( n )} [ i , j ] = ( {K}_{k} * {x}^{( n - 1 )} ) [ i , j ] = {\sum }_{{i}^{′}} {\sum }_{{j}^{′}} {K}_{k} [ {i}^{′} , {j}^{′} ] \cdot {x}^{( n - 1 )} [ i - {i}^{′} , j - {j}^{′} ] .$$

Here the k-th kernel Kₖ is a d-dimensional matrix (in this embodiment a two-dimensional matrix), which is usually small compared to the number of nodes 612-618 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 612-620 in the respective layer 602-610. In particular, for a convolutional layer 604, the number of nodes 614 in the convolutional layer is equivalent to the number of nodes 612 in the preceding layer 602 multiplied with the number of kernels.

If the nodes 612 of the preceding layer 602 are arranged as a d-dimensional matrix, using a plurality of kernels may be interpreted as adding a further dimension (denoted as “depth” dimension), so that the nodes 614 of the convolutional layer 604 are arranged as a (d+1)-dimensional matrix. If the nodes 612 of the preceding layer 602 are already arranged as a (d+1)-dimensional matrix including a depth dimension, using a plurality of kernels may be interpreted as expanding along the depth dimension, so that the nodes 614 of the convolutional layer 604 are arranged also as a (d+1)-dimensional matrix, wherein the size of the (d+1)-dimensional matrix with respect to the depth dimension is by a factor of the number of kernels larger than in the preceding layer 602.

The advantage of using convolutional layers 604 is that spatially local correlation of the input data may exploited by enforcing a local connectivity pattern between nodes of adjacent layers, in particular by each node being connected to only a small region of the nodes of the preceding layer.

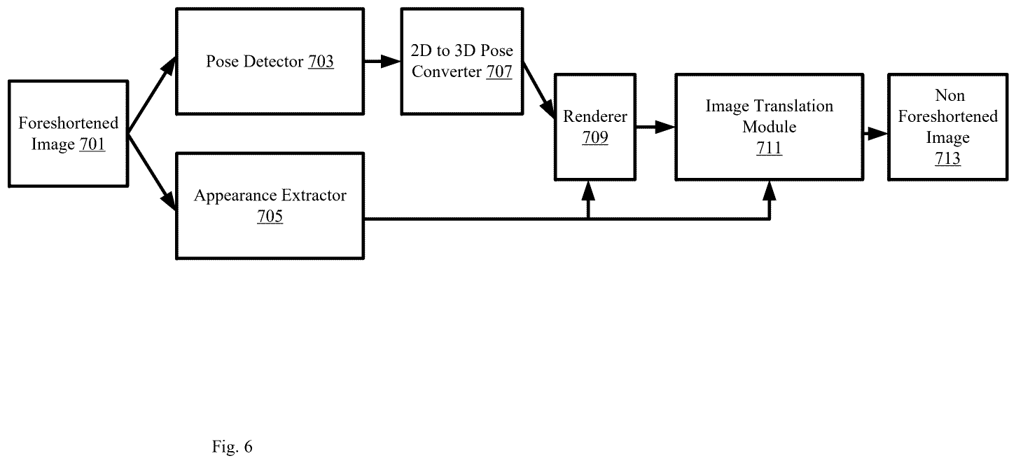

In embodiment shown in FIG. 6, the input layer 602 includes 36 nodes 612, arranged as a two-dimensional 6×6 matrix. The convolutional layer 604 includes 72 nodes 614, arranged as two two-dimensional 6×6 matrices, each of the two matrices being the result of a convolution of the values of the input layer with a kernel. Equivalently, the nodes 614 of the convolutional layer 604 may be interpreted as arranged as a three-dimensional 6×6×2 matrix, wherein the last dimension is the depth dimension.

A pooling layer 606 may be characterized by the structure and the weights of the incoming edges and the activation function of its nodes 616 forming a pooling operation based on a non-linear pooling function f. For example, in the two dimensional case the values x⁽ⁿ⁾ of the nodes 616 of the pooling layer 606 may be calculated based on the values x⁽ⁿ⁻¹⁾ of the nodes 614 of the preceding layer 604 as

$${x}^{( n )} [ i , j ] = f ( {x}^{( n - 1 )} [ {id}_{1} , {jd}_{2} ] , … , {x}^{( n - 1 )} [ {id}_{1} + {d}_{1} - 1 , {jd}_{2} + {d}_{2} - 1 ] )$$

In other words, by using a pooling layer 606, the number of nodes 614, 616 may be reduced, by replacing a number d1·d2 of neighboring nodes 614 in the preceding layer 604 with a single node 616 being calculated as a function of the values of said number of neighboring nodes in the pooling layer. In particular, the pooling function f may be the max-function, the average, or the L2-Norm. In particular, for a pooling layer 606 the weights of the incoming edges are fixed and are not modified by training.

The advantage of using a pooling layer 606 is that the number of nodes 614, 616 and the number of parameters is reduced. This leads to the amount of computation in the network being reduced and to a control of overfitting.

In the embodiment shown in FIG. 6, the pooling layer 606 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 608 may be characterized by the fact that a majority, in particular, all edges between nodes 616 of the previous layer 606 and the nodes 618 of the fully-connected layer 608 are present, and wherein the weight of each of the edges may be adjusted individually.

In this embodiment, the nodes 616 of the preceding layer 606 of the fully-connected layer 608 are displayed both as two-dimensional matrices, and additionally as non-related nodes (indicated as a line of nodes, wherein the number of nodes was reduced for a better presentability). In this embodiment, the number of nodes 618 in the fully connected layer 608 is equal to the number of nodes 616 in the preceding layer 606. Alternatively, the number of nodes 616, 618 may differ.

Furthermore, in this embodiment, the values of the nodes 620 of the output layer 610 are determined by applying the Softmax function onto the values of the nodes 618 of the preceding layer 608. By applying the Softmax function, the sum the values of all nodes 620 of the output layer 610 is 1, and all values of all nodes 620 of the output layer are real numbers between 0 and 1.

A convolutional neural network 600 may also include a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks may be wired using summation (residual/dense neural networks), element-wise multiplication (attention) or other differentiable operators. Therefore, the convolutional neural network architecture may be nested rather than being sequential if the whole pipeline is differentiable.

In particular, convolutional neural networks 600 may be trained based on the backpropagation algorithm. For preventing overfitting, methods of regularization may be used, e.g. dropout of nodes 612-620, stochastic pooling, use of artificial data, weight decay based on the L1 or the L2 norm, or max norm constraints. Different loss functions may be combined for training the same neural network to reflect the joint training objectives. A subset of the neural network parameters may be excluded from optimization to retain the weights pretrained on another datasets.

At act A130, a non-foreshortened image of the heart is generated of the patient by transferring the inferred pose of the heart to a new synthetic view. The appearance of the acquired image may be transferred to the new synthetic view. In an embodiment, Differentiable Rendering (DR) is used to generate the new view. DR includes techniques for end-to-end optimization that obtain useful gradients of the rendering process. An intermediate image may be generated based on the determined pose and the standard view plane. The intermediate image may be generated using the edges and vertices included in the heart model. The intermediate rendered image is feature-based and thus not photorealistic due to a small number (diffuse) of primitives. Increasing the number of primitives may increase the realism, but also the computational cost and the difficulty in optimizing the overall problem. A simplified skeletal structure is thus provided with a high dimensional appearance which is fed to an encoder-decoder network to generate a realistic synthetic view.

FIG. 6 depicts one example of the components and workflow. In an embodiment, the input image 701 is input into the pose detector 703 and the appearance extractor 703. The pose detector 703 detects the pose of the object in the input image. The appearance extractor 703 extracts the appearance of the object. The pose is transferred to a new viewpoint using the 2D to 3D converter 707. The 2D to 3D converter 707 determines the orientation of the slice given the heart model. A new orientation for the slice, for example, a standard A4C view is identified. From the pose seen from a novel orientation, the location and shape of the primitives are derived, which are used, along with their appearance and the intrinsic parameters and distortion coefficients of the new viewpoint, for the rendering 709 of the heart in a high-dimensional image. The output feature image is enhanced in an image translation module 711 to form the output image 713. In an embodiment, the entire workflow may be trained end to end by comparing the output image 713 to a ground truth non foreshortened image. Training data may be synthetically generated using a 3D model and multiple images acquired at different angles.

In an embodiment, various cardiac metrics may be derived from the non-foreshortened view. Examples include chamber volume, ejection fraction, global and longitudinal strain. Ejection fraction, in particular, is very useful in diagnosing cardiac issues. Ejection fraction refers to the amount of blood pumped out of the heart's lower chambers (ventricles) each time it contracts. In the typical heart function, blood enters the heart through the top right section (atrium). Between heartbeats, there's a short pause. This is when blood flows through a valve down to the left ventricle. Once the ventricle is full, the next heartbeat pumps out (ejects) a portion of the blood out to the body. Ejection fraction in a healthy heart is around 50% to 70%. In other words, with each heartbeat, 50% to 70% of the blood in the left ventricle gets pumped out to the rest of the body. Ejection fraction is an indicator of how well the heart is working. A low ejection fraction typically means that the patient has or is at risk for heart failure. The ejection fraction (EF) formula equals the amount of blood pumped out of the ventricle with each contraction (stroke volume or SV) divided by the end-diastolic volume (EDV), the total amount of blood in the ventricle before the contraction.

The apical four-chamber view and apical two-chamber view are used in the primary method (Simpson method) to calculate left ventricle ejection fraction (LVEF). The Simpson Method calculates ejection fraction from the volume of the left ventricle during systole and diastole as estimated from the two 2D views. It is the potentially the best measure of ejection fraction, but it is difficult, time-consuming, and the most operator-dependent technique. The accuracy of the measurement depends on image quality in particular foreshortening which can hamper the volume calculations. In an embodiment, the volume calculations for the Simpson method and others is performed on the synthetic non foreshortened view.

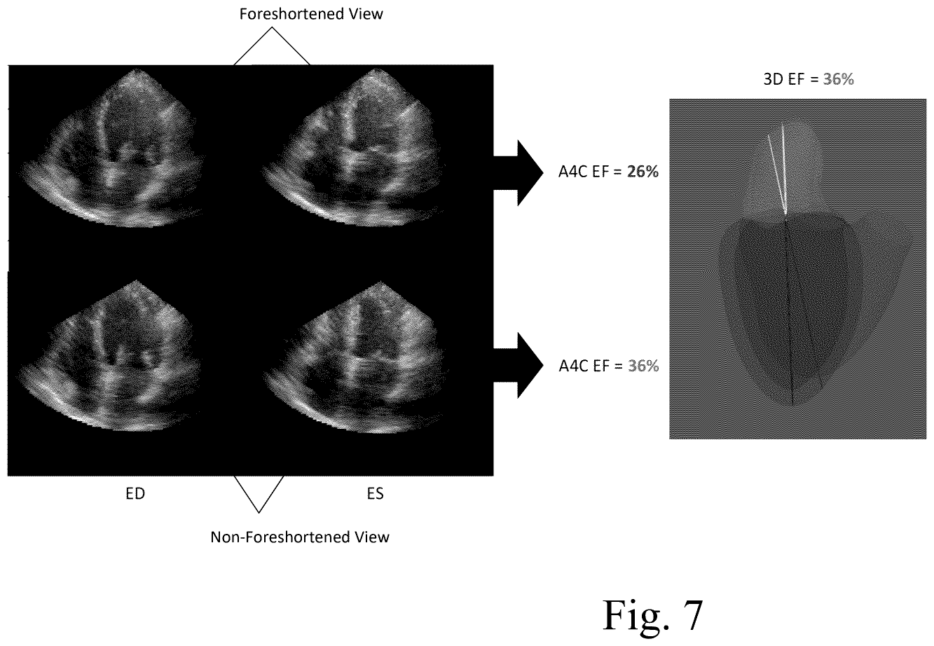

FIG. 7 depicts an example of how EF may be affected by foreshortened views. FIG. 7 depicts End-diastolic (ED) and End-systolic (ES) frames of a foreshortened view (off by 10 degrees) and a non-foreshortened view—as shown in the 3D model. EF for the foreshortened view is computed as 26%. EF for the non-foreshortened view is 36%—as further computed using the 3D model. The measurement error is thus approximately 10%. Both of the computations, for the real acquired image and the synthetic image may be displayed to the operator. A 3D model as depicted on the right may also be provided if available. In an offline workflow, when the method is applied on previously acquired images, the measurement error may be used as an exclusion criterion for images with excessive foreshortening. In that case, if the measurement error exceeds a pre-defined threshold, the image may be excluded from further processing, and for example replaced a synthesized non-foreshortened view based on the input image. The non-foreshortened view may be used instead or in combination with the actual image.

It is to be understood that the elements and features recited in the claims may be combined in different ways to produce new claims that likewise fall within the scope of the present invention. Thus, whereas the dependent claims below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description. Independent of the grammatical term usage, individuals with male, female or other gender identities are included within the term.

The following is a list of non-limiting illustrative embodiments disclosed herein:

Illustrative embodiment 1. A method for automatic correction of view foreshortening in cardiac echo using view synthesis, the method comprising: acquiring, by a medical imaging device, an image representing a foreshortened view of a heart of a patient; inferring, by a processor, a pose of the heart in the acquired image using a trained image segmentation model and a heart model; and generating, by the processor, a non-foreshortened image of the heart of the patient by transferring the inferred pose of the heart to a new synthetic view representing a standard image acquisition plane.

Illustrative embodiment 2. The method according to one of the preceding embodiments, further comprising: applying, by the processor, an appearance of the acquired image to the non-foreshortened image.

Illustrative embodiment 3. The method according to one of the preceding embodiments, wherein the trained image segmentation model is configured to recognize in a given view all visible anatomical structures included in the heart model.

Illustrative embodiment 4. The method according to one of the preceding embodiments, wherein the trained image segmentation model is trained using synthetically generated views and labels generated by intersecting a respective heart model with a plane representing an image acquisition plane.

Illustrative embodiment 5. The method according to one of the preceding embodiments, wherein the foreshortened view comprises a plane that slices the heart of the patient at least 10 degrees offset from a standard view plane.

Illustrative embodiment 6. The method according to one of the preceding embodiments, wherein inferring and generating is performed in real-time with the acquisition of the image.

Illustrative embodiment 7. The method according to one of the preceding embodiments, further comprising: performing measurements of one or more clinically relevant quantities including at least one of chamber volume, ejection fraction, global strain, or longitudinal strain of the heart using the non-foreshortened image.

Illustrative embodiment 8. The method according to illustrative embodiment 7, wherein the measurements are further performed using the acquired image.

Illustrative embodiment 9. The method according to illustrative embodiment 7, wherein the measurements are further performed using the heart model.

Illustrative embodiment 10. The method according to one of the preceding embodiments, wherein the heart model comprises a graph, with N vertices and M edges, wherein the N vertices represent location of junctions between different sections of the heart and the M edges include anatomical structures.

Illustrative embodiment 11. The method according to one of the preceding embodiments, wherein the anatomical structures include at least one of: leaflets of heart valves, and/or segments of each heart chamber, proximal portions of arteries and veins attached to the heart.

Illustrative embodiment 12. The method according to one of the preceding embodiments, wherein the standard image acquisition plane comprises an apical-four-chamber view without foreshortening.

Illustrative embodiment 13. The method according to illustrative embodiment 12, further comprising: generating instructions for placement of a transducer probe to acquire a real non-foreshortened image of the heart of the patient, the instructions based on a difference between the non-foreshortened image of the heart of the patient and the acquired foreshortened image of the heart of the patient.

Illustrative embodiment 14. A system for automatic correction of view foreshortening in cardiac echo using view synthesis, the system comprising: an ultrasound probe configured to acquire an image of a cardiac region of a patient; an imaging processor configured to generate a non-foreshortened image of the cardiac region of the patient using novel view synthesis, the acquired image, and a heart model; and a display configured to display the non-foreshortened image.

Illustrative embodiment 15. The system according to one of the preceding embodiments, wherein a heart is modeled as a graph, with N vertices and M edges, wherein the N vertices represent location of junctions between different sections of the heart and the M edges includes anatomical structures.

Illustrative embodiment 16. The system according to one of the preceding embodiments, wherein novel view synthesis comprises at least using a trained segmentation network to identify and match visible anatomical structures in the acquired image to anatomical structures in the heart model.

Illustrative embodiment 17. The system according to one of the preceding embodiments, wherein the acquired image is acquired with at least a 10 degree offset from a standard imaging plane.

Illustrative embodiment 18. The system according to one of the preceding embodiments, wherein the imaging processor is further configured to compute one or more clinically relevant quantities including at least one of chamber volume, ejection fraction, global strain, or longitudinal strain of a heart using the non-foreshortened image.

Illustrative embodiment 19. The system according to one of the preceding embodiments, wherein the acquired image is foreshortened and wherein the imaging processor is further configured to define modifications required in an acquisition plane to modify an actual view for acquiring the acquired image to a new view for acquiring a real non-foreshortened image.

Illustrative embodiment 20. A non-transitory computer implemented storage medium that stores machine-readable instructions executable by at least one processor for correction of view foreshortening in cardiac echo using view synthesis, the machine-readable instructions comprising: acquiring a foreshortened image of a cardiac region of a patient using a first view; determining a pose of one or more features of a heart of the patient in the foreshortened image; transferring the pose of the one or more features to a new view of the cardiac region, the new view from a different angle than the first view; and generating a non-foreshortened image from the new view.

1. A method for automatic correction of view foreshortening in cardiac echo using view synthesis, the method comprising:
acquiring, by a medical imaging device, an image representing a foreshortened view of a heart of a patient;
inferring, by a processor, a pose of the heart in the acquired image using a trained image segmentation model and a heart model; and
generating, by the processor, a non-foreshortened image of the heart of the patient by transferring the inferred pose of the heart to a new synthetic view representing a standard image acquisition plane.

2. The method of claim 1, further comprising
applying, by the processor, an appearance of the acquired image to the non-foreshortened image.

3. The method of claim 1, wherein the trained image segmentation model is configured to recognize in a given view all visible anatomical structures included in the heart model.

4. The method of claim 1, wherein the trained image segmentation model is trained using synthetically generated views and labels generated by intersecting a respective heart model with a plane representing an image acquisition plane.

5. The method of claim 1, wherein the foreshortened view comprises a plane that slices the heart of the patient at least 10 degrees offset from a standard view plane.

6. The method of claim 1, wherein inferring and generating is performed in real-time with the acquisition of the image.

7. The method of claim 1, further comprising:
performing measurements of one or more clinically relevant quantities including at least one of chamber volume, ejection fraction, global strain, or longitudinal strain of the heart using the non-foreshortened image.

8. The method of claim 7, wherein the measurements are further performed using the acquired image.

9. The method of claim 7, wherein the measurements are further performed using the heart model.

10. The method of claim 1, wherein the heart model comprises a graph, with N vertices and M edges, wherein the N vertices represent location of junctions between different sections of the heart and the M edges include anatomical structures.

11. The method of claim 10, wherein the anatomical structures include at least one of: leaflets of heart valves, and/or segments of each heart chamber, proximal portions of arteries and veins attached to the heart.

12. The method of claim 1, wherein the standard image acquisition plane comprises an apical-four-chamber view without foreshortening.

13. The method of claim 12, further comprising:
generating instructions for placement of a transducer probe to acquire a real non-foreshortened image of the heart of the patient, the instructions based on a difference between the non-foreshortened image of the heart of the patient and the acquired foreshortened image of the heart of the patient.

14. A system for automatic correction of view foreshortening in cardiac echo using view synthesis, the system comprising:
an ultrasound probe configured to acquire an image of a cardiac region of a patient;
an imaging processor configured to generate a non-foreshortened image of the cardiac region of the patient using novel view synthesis, the acquired image, and a heart model; and
a display configured to display the non-foreshortened image.

15. The system of claim 14, wherein a heart is modeled as a graph, with N vertices and M edges, wherein the N vertices represent location of junctions between different sections of the heart and the M edges includes anatomical structures.

16. The system of claim 15, wherein novel view synthesis comprises at least using a trained segmentation network to identify and match visible anatomical structures in the acquired image to anatomical structures in the heart model.

17. The system of claim 14, wherein the acquired image is acquired with at least a 10 degree offset from a standard imaging plane.

18. The system of claim 14, wherein the imaging processor is further configured to compute one or more clinically relevant quantities including at least one of chamber volume, ejection fraction, global strain, or longitudinal strain of a heart using the non-foreshortened image.

19. The system of claim 14, wherein the acquired image is foreshortened and wherein the imaging processor is further configured to define modifications required in an acquisition plane to modify an actual view for acquiring the acquired image to a new view for acquiring a real non-foreshortened image.

20. A non-transitory computer implemented storage medium that stores machine-readable instructions executable by at least one processor for correction of view foreshortening in cardiac echo using view synthesis, the machine-readable instructions comprising:
acquiring a foreshortened image of a cardiac region of a patient using a first view;
determining a pose of one or more features of a heart of the patient in the foreshortened image;
transferring the pose of the one or more features to a new view of the cardiac region, the new view from a different angle than the first view; and
generating a non-foreshortened image from the new view.
